(19) United States
(12) Patent Application Publication
(10) Pub. No.: US 2021/0356391 A1
(43) Pub. Date: Nov. 18, 2021

(54) SYSTEMS AND METHODS FOR TUMOR SUBTYPING USING MOLECULAR CHEMICAL IMAGING

(71) Applicant: CHEMIMAGE CORPORATION, Pittsburgh, PA (US)

(72) Inventors: Shona STEWART, Pittsburgh, PA (US); Heather GOMER, Sewickley, PA (US); Arash SAMIEI, Pittsburgh, PA (US); Marlena DARR, McKeesport, PA (US)

(21) Appl. No.: 17/321,206

(22) Filed: May 14, 2021

Related U.S. Application Data
(60) Provisional application No. 63/025,467, filed on May 15, 2020.

(51) Int. Cl. G01N 21/31; G01N 21/21; G01N 21/25; G01N 21/33; G01N 21/3581; G01N 21/359; G01N 33/483

(52) U.S. Cl. CPC G01N 21/314; G01N 21/21; G01N 21/255; G01N 33/4833; G01N 21/3581; G01N 21/359; G01N 21/33

(57) ABSTRACT

Systems and methods designed to determine tumor histological subtypes in order to guide a surgical procedure. The systems and methods illuminate biological tissue in order to generate a plurality of interacted photons, collect the interacted photons, detect the plurality of a interacted photons to generate at least one hyperspectral image, and analyze a hyperspectral image by extracting a spectrum from a location in the hyperspectral image. The location should correspond to an area that is of interest in the biological tissue.

### CROSS-REFERENCE TO RELATED APPLICATIONS

The application claims the benefit of U.S. Provisional Patent Application No. 63/025,467 filed May 15, 2020, the entirety of which is incorporated by reference herein.

### FIELD

The present disclosure pertains to systems and methods for identifying cancer histological subtypes. More particularly, the present disclosure pertains to systems and methods of identifying and differentiating among cancer histological subtypes using molecular chemical imaging or hyperspectral imaging.

### BACKGROUND

Cancer is an enormous global health burden, accounting for one in every eight deaths worldwide. A critical problem in cancer management is the local recurrence of disease, which is often a result of incomplete excision of tumor cells. Currently, the presence of tumor cells at the surgical margins must be identified through histological evaluation in a pathology lab. Approximately one in four patients who undergo tumor resection surgery will require re-operation in order to fully excise the malignant tissue. Recent efforts aimed towards significantly reducing the frequency of local recurrence have employed diffuse reflectance, radiofrequency spectroscopy, and targeted fluorescence imaging.

Current techniques for gross anatomic pathology require inspection by a pathologist and are therefore inherently subjective. As such, there exists a need for a system and method that would enable objective analysis of tissue samples in order to improve the accuracy of pathological determinations. In particular, it would be advantageous if the system and method could be used to assess a variety of characteristics of a sample including anatomical features, detect cancerous tissue, and locate the presence of a tumor at the surgical margins.

In addition, conventional surgical techniques do not allow a surgeon to intraoperatively identify different histological subtypes of tumors. It would be beneficial to determine tumor histological subtypes intraoperatively in order to guide a surgical procedure. Determination of tumor histological subtypes may also be useful in defining follow up treatment for the patient. As such, a need exists for methods and systems of determining histological subtypes of cancerous tissue.

### SUMMARY

Systems and methods for analyzing biological tissues, such as organs or skin are disclosed.

In one embodiment, there is method of analyzing biological tissue, the method comprising: illuminating the biological tissue to generate a plurality of interacted photons; collecting the plurality of interacted photons; detecting the plurality of interacted photons to generate at least one hyperspectral image; analyzing the at least one hyperspectral image by extracting a spectrum from a location in the at least one hyperspectral image, wherein the location corresponds to an area of interest of the biological tissue; and analyzing the extracted spectrum to differentiate a tumor histological subtype present within the biological tissue.

In another embodiment, the biological tissue comprises tissue from one or more of a kidney, a ureter, a prostate, a penis, a testicle, a bladder, a heart, a brain, a liver, a lung, a colon, an intestine, a pancreas, a thyroid, an adrenal gland, a spleen, a stomach, a uterus, and an ovary.

In another embodiment, the tumor histological subtype comprises a histological subtype of one or more of kidney cancer, bladder cancer, bone cancer, brain cancer, breast cancer, colon cancer, intestinal cancer, liver cancer, lung cancer, ovarian cancer, pancreatic cancer, prostate cancer, rectal cancer, skin cancer, stomach cancer, testicular cancer, thyroid cancer, urethral cancer, and uterine cancer.

In another embodiment, the method further comprises generating a bright-field image representative of the biological tissue.

In another embodiment, the method further comprises analyzing the bright-field image to identify one or more of a morphological feature of the biological tissue and an anatomical feature of the biological tissue.

In another embodiment, analyzing the extracted spectrum further comprises comparing the extracted spectrum to a reference spectrum associated with a known characteristic.

In another embodiment, the comparing comprises applying an algorithmic technique.

In another embodiment, the algorithmic technique comprises one or more of a multivariate curve resolution analysis, a principle component analysis (PCA), a partial least squares discriminant analysis (PLSDA), a non-negative matrix factorization, a k means clustering analysis, a band target entropy method analysis, an adaptive subspace detector analysis, a cosine correlation analysis, a Euclidian distance analysis, a partial least squares regression analysis, a spectral mixture resolution analysis, a spectral angle mapper metric analysis, a spectral information divergence metric analysis, a Mahalanobis distance metric analysis, and a spectral unmixing analysis.

In another embodiment, the algorithmic technique comprises one or more of a support vector machine and a relevance vector machine.

In another embodiment, the algorithmic technique is applied to spectra corresponding to each pixel of the at least one hyperspectral image to generate at least one score image.

In another embodiment, the at least one score image comprises one or more of a target image and a non-target image.

In another embodiment, the method further comprises applying a threshold to the target image to generate a class image of the biological tissue.

In another embodiment, the method further comprises generating an RGB image of the biological tissue, wherein at least one channel of the RGB image corresponds to the target image.

In another embodiment, the method comprises generating an RGB image of the biological tissue, wherein at least one channel of the RGB image corresponds to a non-target image.

In another embodiment, the hyperspectral image comprises a VIS-NIR hyperspectral image.

In another embodiment, the hyperspectral image comprises a SWIR hyperspectral image.

In another embodiment, the method comprises passing the plurality of interacted photons through a filter to filter the interacted photons across a plurality of wavelength bands.

In one embodiment, there is a system for analyzing biological tissue, the system comprising one or more processors coupled to a non-transitory processor-readable medium, the non-transitory processor-readable medium including instructions that, when executed by the one or more processors, cause the system to: illuminate the biological tissue to generate a plurality of interacted photons; collect the plurality of interacted photons; detect the plurality of interacted photons to generate at least one hyperspectral image; analyze the at least one hyperspectral image by extracting a spectrum from a location in the at least one hyperspectral image, wherein the location corresponds to an area of interest of the biological tissue; and analyze the extracted spectrum to differentiate a tumor histological subtype present within the biological tissue.

In another embodiment, the biological tissue comprises tissue from one or more of a kidney, a ureter, a prostate, a penis, a testicle, a bladder, a heart, a brain, a liver, a lung, a colon, an intestine, a pancreas, a thyroid, an adrenal gland, a spleen, a stomach, a uterus, and an ovary.

In another embodiment, the tumor histological subtype comprises a histological subtype of one or more of kidney cancer, bladder cancer, bone cancer, brain cancer, breast cancer, colon cancer, intestinal cancer, liver cancer, lung cancer, ovarian cancer, pancreatic cancer, prostate cancer, rectal cancer, skin cancer, stomach cancer, testicular cancer, thyroid cancer, urethral cancer, and uterine cancer.

In another embodiment, the instructions, when executed by the one or more processors, further cause the system to generate a bright-field image representative of the biological tissue.

In another embodiment, the instructions, when executed by the one or more processors, further cause the system to analyze the bright-field image to identify one or more of a morphological feature of the biological tissue and an anatomical feature of the biological tissue.

In another embodiment, the instructions, when executed by the one or more processors, further cause the system to compare the extracted spectrum to a reference spectrum associated with a known characteristic.

In another embodiment, the comparing comprises applying an algorithmic technique.

In another embodiment, the algorithmic technique comprises one or more of a multivariate curve resolution analysis, a principle component analysis (PCA), a partial least squares discriminant analysis (PLSDA), a non-negative matrix factorization, a k means clustering analysis, a band target entropy method analysis, an adaptive subspace detector analysis, a cosine correlation analysis, a Euclidian distance analysis, a partial least squares regression analysis, a spectral mixture resolution analysis, a spectral angle mapper metric analysis, a spectral information divergence metric analysis, a Mahalanobis distance metric analysis, and a spectral unmixing analysis.

In another embodiment, the algorithmic technique comprises one or more of a support vector machine and a relevance vector machine.

In another embodiment, the instructions, when executed by the one or more processors, further cause the system to apply the algorithmic technique to spectra corresponding to each pixel of the at least one hyperspectral image to generate at least one score image.

In another embodiment, the at least one score image comprises one or more of a target image and a non-target image.

In another embodiment, the instructions, when executed by the one or more processors, further cause the system to apply a threshold to the target image to generate a class image of the biological tissue.

In another embodiment, the instructions, when executed by the one or more processors, further cause the system to generate an RGB image of the biological tissue, wherein at least one channel of the RGB image corresponds to the target image.

In another embodiment, the instructions, when executed by the one or more processors, further cause the system to generate an RGB image of the biological tissue, wherein at least one channel of the RGB image corresponds to a non-target image.

In another embodiment, the hyperspectral image comprises a VIS-NIR hyperspectral image.

In another embodiment, the hyperspectral image comprises a SWIR hyperspectral image.

In another embodiment, the instructions, when executed by the one or more processors, further cause the system to pass the plurality of interacted photons through a filter to filter the interacted photons across a plurality of wavelength bands.

### DRAWINGS

The accompanying drawings, which are included to provide further understanding of the disclosure and are incorporated in and constitute a part of this specification, illustrate embodiments of the disclosure and, together with the description, serve to explain the principles of the disclosure.

FIG. 1 depicts a block diagram of an illustrative environment with an exemplary tissue detecting computing device in accordance with an embodiment.

FIG. 2 depicts a block diagram of an exemplary tissue detecting computing device in accordance with an embodiment.

FIG. 3 depicts a flow diagram of an illustrative method of detecting tumor histological subtypes in accordance with an embodiment.

FIG. 4 depicts average VIS-NIR spectra for a plurality of kidney cancer tumor histological subtypes used in a multi-class discriminant analysis.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

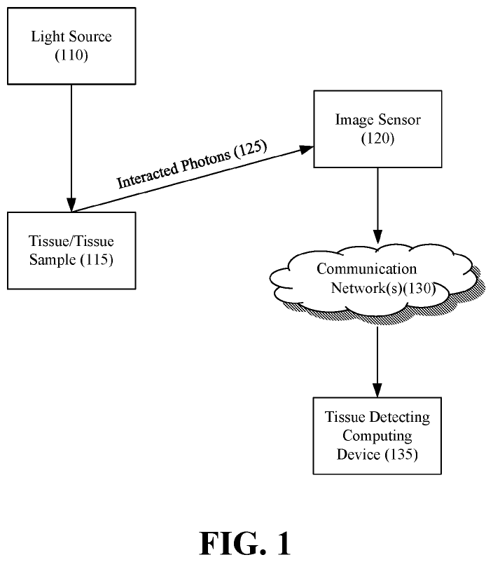

Referring to FIG. 1, an illustrative environment with an exemplary tissue detecting computing device is depicted. The environment includes a light source 110 configured to generate photons to illuminate tissue 115 (or a tissue sample), an image sensor 120 positioned to collect interacted photons 125, and a tissue detecting computing device 130 coupled to the image sensor via one or more communication networks 130, although the environment can include other types and/or numbers of devices or systems coupled in other manners, such as additional server devices. This technology provides a number of advantages including providing methods, non-transitory computer readable media, and tissue detecting computing devices that provide the ability to determine the histological subtype of a particular tumor. In particular, certain implementations of this technology provide a real-time, non-contact method for determining tumor histological subtypes during a surgical procedure in order to direct the surgical plan and post-operative treatment.

### Light Source

In an embodiment, at least one light source 110 generates photons that are directed to tissue 115 in a human or an animal. The at least one light source 110 is not limited by this disclosure and can be any source that is useful in providing illumination. In an embodiment, the at least one light source 110 may be used in concert with or attached to endoscope. Other ancillary requirements, such as power consumption, emitted spectra, packaging, thermal output, and so forth may be determined based on the particular application for which the at least one light source 110 is used. In some embodiments, the at least one light source 110 comprises a light element, which is an individual device that emits light. The types of light elements are not limited and may include an incandescent lamp, halogen lamp, light emitting diode (LED), chemical laser, solid state laser, organic light emitting diode (OLED), electroluminescent device, fluorescent light, gas discharge lamp, metal halide lamp, xenon arc lamp, induction lamp, quantum dot, or any combination of these light sources. In other embodiments, the at least one light source 110 is a light array, which is a grouping or assembly of a plurality of light elements that are placed in proximity to each other.

In some embodiments, the at least one light source 110 has a particular wavelength that is intrinsic to the light element or to the light array. In other embodiments, the wavelength of the at least one light source 110 may be modified by filtering or tuning the photons that are emitted by the light source. In still other embodiments, light sources 110 having different wavelengths are combined. In one embodiment, the selected wavelength of the at least one light source 110 is in the visible-near infrared (VIS-NIR) or shortwave infrared (SWIR) ranges. These correspond to wavelengths of about 400 nm to about 1100 nm (VIS-NIR), or about 850 nm to about 1800 nm (SWIR). The above ranges may be used alone or in combination with any of the listed ranges or other wavelength ranges. Such combinations include adjacent (contiguous) ranges, overlapping ranges, and ranges that do not overlap.

In some embodiments, the at least one light source 110 comprises a modulated light source. The choice of a modulated light source 110 and the techniques for modulating the light source are not limited. In some embodiments, the modulated light source 110 is one or more of a filtered incandescent lamp, filtered halogen lamp, tunable LED array, tunable solid state laser array, tunable OLED array, tunable electroluminescent device, filtered fluorescent light, filtered gas discharge lamp, filtered metal halide lamp, filtered xenon arc lamp, filtered induction lamp, quantum dot, or any combination of these light sources. In some embodiments, tuning is accomplished by increasing or decreasing the intensity or duration at which individual light elements 110 are powered. In some embodiments, tuning is accomplished by a fixed or tunable filter (not shown) that filters light emitted by individual light elements. In still other embodiments, the at least one light source 110 is not tunable. A light source 110 that is not tunable cannot change its emitted light spectra, but it can be turned on and off by appropriate controls.

In some embodiments, imaging may be performed by filtering and detecting interacted photons 125 that are reflected from the tissue 115 of the human or animal patient (or a tissue sample) using the image sensor 120 and associated optics, such as filters. The image sensor 120 can be any suitable image sensor for molecular chemical imaging (MCI). The techniques and devices for filtering are not limited and include any of fixed filters, multi-conjugate filters, and conformal filters. In fixed filters, the functionality of the filter cannot be changed, though the filtering can be changed by mechanically moving the filter into or out of the light path. In some embodiments, real-time image detection is employed using a dual polarization configuration using either multi-conjugate filters or conformal filters. In some embodiments, the filter is a tunable filter that comprises a multi-conjugate filter. The multi-conjugate filter is an imaging filter with serial stages along an optical path in a Solc filter configuration. In such filters, angularly distributed retarder elements of equal birefringence are stacked in each stage with a polarizer between stages.

A conformal filter can filter a broadband spectra into one or more passbands. Example conformal filters include a liquid crystal tunable filter, an acousto-optical tunable filter, a Lyot liquid crystal tunable filter, an Evans Split-Element liquid crystal tunable filter, a Solc liquid crystal tunable filter, a Ferroelectric liquid crystal tunable filter, a Fabry Perot liquid crystal tunable filter, and combinations thereof.

In an embodiment, the image sensor 120 comprises a camera chip. The camera chip 120 is not limited; however, in some embodiments, the camera chip is selected depending on the expected spectra that is reflected from the tissues of the human or animal patient. The tissues can include one or more of skin or organs. In some embodiments, the camera chip 120 is one or more of a charge coupled device (CCD), a complementary metal oxide semiconductor (CMOS), an indium gallium arsenide (InGaAs) camera chip, a platinum silicide (PtSi) camera chip, an indium antimonide (InSb) camera chip, a mercury cadmium telluride (HgCdTe) camera chip, or a colloidal quantum dot (CQD) camera chip. In some embodiments, each or a combination of the above-listed camera chips 120 is a focal plane array (FPA). In some embodiments, any of the above-listed camera chips 120 may include quantum dots to tune their bandgaps, thereby altering or expanding sensitivity to different wavelengths. The visualization techniques are not limited, and include one or more of VIS, NIR, SWIR, autofluorescence, or Raman spectroscopy. Although the image sensor 120 is illustrated as a standalone device, the image sensor could be incorporated in the tissue detecting computing device 135 or in a device associated with the light source 110.

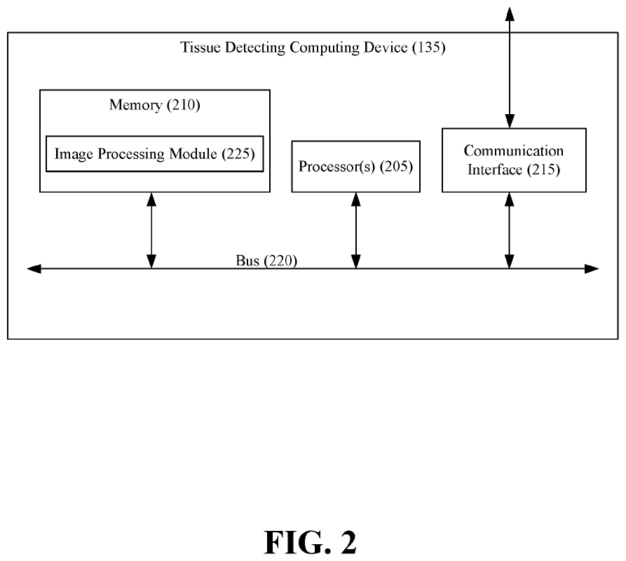

Referring to FIGS. 1-2, the tissue detecting computing device 135 in this example includes one or more processors 205, one or more memories 210, and/or a communication interface 215, which are coupled together by a bus 220 or other communication link, although the tissue detecting computing device can include other types and/or numbers of elements in other configurations. The one or more processors 205 of the tissue detecting computing device 135 may execute programmed instructions stored in the memory 210 for any number of the functions described and illustrated herein. The one or more processors 205 of the tissue detecting computing device 135 may include one or more CPUs or general purpose processors with one or more processing cores, for example, although other types of processors can also be used.

The memory 210 of the tissue detecting computing device may store the programmed instructions for one or more aspects of the present technology as described and illustrated herein, although some or all of the programmed instructions could be stored elsewhere. A variety of different types of memory storage devices, such as random access memory (RAM), read only memory (ROM), hard disk, solid state drives, flash memory, or other computer readable media that are read from and written to by a magnetic, optical, or other reading and writing system that is coupled to the one or more processors 205, can be used for the memory 210.

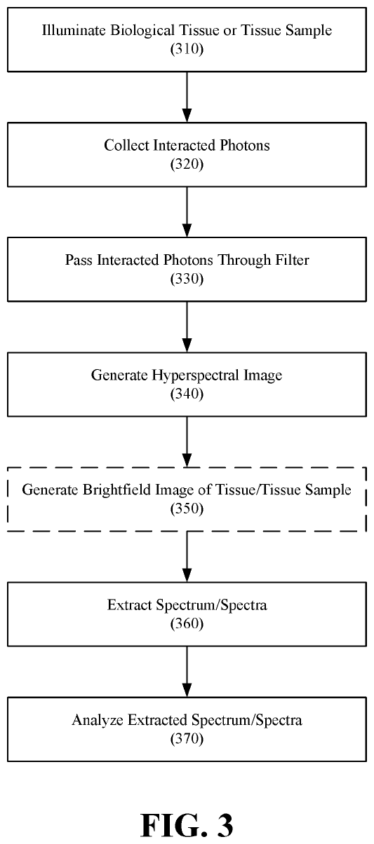

Accordingly, the memory 210 of the tissue detecting computing device 135 can store one or more applications that include executable instructions that, when executed by the one or more processors 205, cause the tissue detecting computing device to perform actions, such as to perform the actions described and illustrated below with reference to FIG. 3. The one or more applications can be implemented as modules or components of other applications. Further, the one or more applications can be implemented as operating system extensions, modules, plugins, or the like.

In some embodiments, the one or more applications may be operative in a cloud-based computing environment. In some embodiments, the one or more applications may be executed within or as one or more virtual machines or one or more virtual servers that may be managed in a cloud-based computing environment. In some embodiments, the one or more applications, and even the tissue detecting computing device 135 itself, may be located in one or more virtual servers running in a cloud-based computing environment rather than being tied to one or more specific physical network computing devices. In some embodiments, the one or more applications may be running in one or more virtual machines (VMs) executing on the tissue detecting computing device 135. Additionally, in some embodiments of this technology, one or more virtual machines running on the tissue detecting computing device 135 may be managed or supervised by a hypervisor.

In this particular example, the memory 210 of the tissue detecting computing device 135 includes an image processing module 225, although the memory can include other policies, modules, databases, or applications, for example. The image processing module 225 in this example is configured to analyze image data from the image sensor 120 to identify whether a tissue 115 comprises cancerous tissue and/or to determine a type of cancerous tissue based on the image data, although the image processing module could perform other functions in addition to these operations. By way of example only, the image processing module 225 may apply one or more machine learning techniques such as image weighted Bayesian function, logistic regression, linear regression, regression with regularization, naïve Bayes, classification and regression trees (CART), support vector machines, or a neural network to process the image data. In some embodiments, the image processing module 225 may apply a multivariate analytical technique, such as support vector machines (SVM) and/or relevance vector machines (RVM). In some embodiments, the image processing module 225 may apply at least one chemometric technique. Illustrative chemometric techniques that the image processing module 225 may apply include, but are not limited to: multivariate curve resolution, principle component analysis (PCA), partial least squares discriminant analysis (PLSDA), a non-negative matrix factorization, k means clustering, band-target entropy method (BTEM), adaptive subspace detector, cosine correlation analysis, Euclidian distance analysis, partial least squares regression, spectral mixture resolution, a spectral angle mapper metric, a spectral information divergence metric, a Mahalanobis distance metric, and spectral unmixing.

The communication interface 215 of the tissue detecting computing device 135 operatively couples and communicates between the tissue detecting computing device, the image sensor 120, the additional sensors, the client devices and/or the server devices, which are all coupled together by the one or more communication networks 130, although other types and/or numbers of communication networks or systems with other types and/or numbers of connections and/or configurations to other devices and/or elements can also be used.

By way of example only, the one or more communication networks 130 shown in FIG. 1 can include one or more local area networks (LANs) and/or one or more wide area networks (WANs). In some embodiments, the one or more communication networks 130 may use TCP/IP over Ethernet and industry-standard protocols, although other types and/or numbers of protocols and/or communication networks can be used. The one or more communication networks 130 in this example can employ any suitable interface mechanisms and network communication technologies including, for example, teletraffic in any suitable form (e.g., voice, modem, and the like), Public Switched Telephone Networks (PSTNs), Ethernet-based Packet Data Networks (PDNs), combinations thereof, and the like.

The tissue detecting computing device 135 can be a standalone device or integrated with one or more other devices or apparatuses, such as the image sensor or the one or more of the server devices or the client devices, for example. In one particular example, the tissue detecting computing device 135 can include or be hosted by one of the server devices or one of the client devices, and other arrangements are also possible.

Although the exemplary environment with the tissue detecting computing device 135, at least one light source 110, image sensor 120, and one or more communication networks 130 are described and illustrated herein, other types and/or numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art.

One or more of the devices depicted in the environment, such as the tissue detecting computing device 135, for example, may be configured to operate as virtual instances on the same physical machine. In other words, one or more of the tissue detecting computing device 135, client devices, or server devices may operate on the same physical device rather than as separate devices communicating through one or more communication networks. Additionally, there may be more or fewer tissue detecting computing devices 135 than illustrated in FIG. 1.

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on one or more computer systems that extend across any suitable network using any suitable interface mechanisms and traffic technologies, including by way of example only wireless networks, cellular networks, PDNs, the Internet, intranets, and combinations thereof.

The examples may also be embodied as one or more non-transitory computer readable media (e.g., memory 210 ) having instructions stored thereon for one or more aspects of the present technology as described and illustrated by way of the examples herein. The instructions in some examples include executable code that, when executed by one or more processors (e.g., the one or more processors 205 ), cause the one or more processors to carry out steps necessary to implement the methods of the examples of this technology that are described and illustrated herein.

An illustrative method of tumor histological subtype detection will now be described with reference to FIG. 3. The tissue detecting computing device collects image data from the image sensor. In some embodiments, the image data can be hyperspectral image data. In some embodiments, the image sensor is positioned to collect interacted photons from a tissue region resulting from illumination of the tissue sample at a plurality of wavelengths using the light source. In one example, the light source is located on an endoscopic device. In some embodiments, the light source illuminates the tissue region using wavelengths in the visible near infrared (VIS-NIR) and/or shortwave infrared (SWIR) regions.

The present disclosure also provides for a method for analyzing tissue samples, such as biological tissue sample or organ samples, using hyperspectral imaging. The present disclosure contemplates a variety of organ types may be analyzed using the system and method provided herein, including but not limited to: a kidney, a ureter, a prostate, a penis, a testicle, a bladder, a heart, a brain, a liver, a lung, a colon, an intestine, a pancreas, a thyroid, an adrenal gland, a spleen, a stomach, a uterus, and an ovary.

In one embodiment, illustrated by FIG. 3, at least a portion of biological tissue or a biological tissue sample may be illuminated 310 to generate at least one plurality of interacted photons. In some embodiments, the biological tissue may be illuminated 310 in vivo during, for example, a surgical procedure. In some embodiments, the biological tissue sample may be illuminated 310 ex vivo as part of a biopsy/histopathology analysis. The interacted photons may comprise photons absorbed by the biological tissue, photons reflected by the biological tissue, photons scattered by the biological tissue, and photons emitted by the biological tissue.

The interacted photons may be collected 320 and passed 330 through at least one filter to filter the interacted photons into a plurality of wavelength bands. In some embodiments, the at least one filter may comprise a fixed filter (such as a thin film fixed bandpass filter) and/or a tunable filter.

The filtered photons may be detected and at least one hyperspectral image may be generated 340. The at least one hyperspectral image may be representative of the biological tissue. In some embodiments, the hyperspectral image may comprise at least one VIS-NIR hyperspectral image. In some embodiments, the hyperspectral image may comprise at least one SWIR hyperspectral image. In some embodiments, each pixel of the image may comprise at least one spectrum representative of the biological material at that location in the biological tissue.

In some embodiments, the method may further comprise the use of dual polarization. In such an embodiment, the interacted photons may be separated into two orthogonally-polarized components (i.e., photons corresponding to a first optical component and photons corresponding to a second optical component). The first optical component may be transmitted to a first filter, and the second optical component may be transmitted to a second filter. The photons associated with each component may be filtered by the corresponding filter to generate filtered photons. In one embodiment, filtered photons corresponding to a first optical component may be detected by a first detector and filtered photons corresponding to a second optical component may be detected by a second detector. In some embodiments, hyperspectral images may be overlaid on a display. In some embodiments, hyperspectral images may be displayed adjacent to each other or in any other configuration. In some embodiments, the filtered photons may be detected simultaneously. In some embodiments, the filtered photons may be detected sequentially.

In one embodiment, a bright-field image of the biological tissue may be generated. The present disclosure contemplates that any of several methods may be used to generate a bright-field image which would not require further configuration of a detector. In one embodiment, a reflectance hypercube can be generated and contracted. A plurality of frames corresponding to a desired wavelength range may be extracted from the hypercube using ChemImage Xpert® software, available from ChemImage Corporation, Pittsburgh, Pa. In one embodiment, the range may comprise at least one of: about 400 nm to about 710 nm and about 380 nm to about 700 nm. Such software may convert a visible hyperspectral image into a bright-field image using a Wavelength Color Transform (WCT) function. The WCT function may apply red, green, and blue coloration, proportionate to pixel intensity, to the frames for wavelengths in ranges of about 610 nm to about 710 nm, about 505 nm to about 605 nm, and about 400 nm to about 500 nm, respectively. As a result, an RGB (WCT) image may be derived from the hypercube.

The bright-field image may be further analyzed and/or annotated to assess various features such as morphological features and/or anatomic features. In addition, the present disclosure also contemplates traditional digital images may be obtained of the biological tissue for annotation and to aid in analysis. This annotation may be performed by a surgeon, pathologist, or other clinician.

Referring back to FIG. 3, at least one spectrum may be extracted 360 from at least one location corresponding to a region of interest of the biological tissue. In some embodiments, a plurality of spectra from a plurality of locations may be extracted 360, wherein each location corresponds to a region of interest of the biological tissue. For example, in some embodiments, a plurality of spectra may be extracted 360 from the hyperspectral image at a location corresponding to a region of the biological tissue suspected to be a cancerous tumor, and a plurality of spectra may be extracted from the hyperspectral image at a location corresponding to a region of the biological tissue suspected to be non-cancerous (i.e., normal tissue). In another embodiment, spectra may be extracted 360 from various locations of a tissue or an organ to help identify various anatomical features and/or tissue margins. In some embodiments, the biological tissue may correspond to a tumor histological subtype. For example, the tumor histological subtype may include one or more of a histological subtype of kidney cancer, bladder cancer, bone cancer, brain cancer, breast cancer, colon cancer, intestinal cancer, liver cancer, lung cancer, ovarian cancer, pancreatic cancer, prostate cancer, rectal cancer, skin cancer, stomach cancer, testicular cancer, thyroid cancer, urethral cancer, or uterine cancer.

The extracted spectra may be analyzed 370 to assess at least one characteristic of the biological tissue, such as a tumor histological subtype. In one embodiment, the present disclosure contemplates analyzing 360 the spectra by applying at least one algorithm. In some embodiments, supervised classification of the data may be achieved by applying a multivariate analytical technique, such as support vector machines (SVM) and/or relevance vector machines (RVM). In some embodiments, the present disclosure contemplates that the algorithm may comprise at least one chemometric technique. Illustrative chemometric techniques that may be applied include, but are not limited to: multivariate curve resolution, principle component analysis (PCA), partial least squares discriminant analysis (PLSDA), a non-negative matrix factorization, k means clustering, band-target entropy method (BTEM), adaptive subspace detector, cosine correlation analysis, Euclidian distance analysis, partial least squares regression, spectral mixture resolution, a spectral angle mapper metric, a spectral information divergence metric, a Mahalanobis distance metric, and spectral unmixing.

Embodiments applying PLSDA are described hereinbelow. In such embodiments, a PLSDA prediction outcome may include a probability value between zero and one, where one indicates membership within a class, and zero indicates non-membership within a class.

In some embodiments, a traditional two-class model may be used to assess two characteristics of the biological tissue. Examples of characteristics analyzed using a two-class model may include, but are not limited, to: tumor v. non-tumor, cancer v. non-cancer, and specific anatomical features v. features comprising the remainder of the biological sample. As used herein, characteristics analyzed using a two-class model may further include a first tumor histological subtype v. a second tumor histological subtype.

In a two-class model, extracted spectra and/or reference spectra may be selected for each class. The spectra may be pre-processed by applying techniques such as spectral truncation (for example, in a range between about 560 nm and about 1035 nm), baseline subtraction, zero offset, and vector normalization. A leave one patient out (LOPO) PLSDA analysis may be applied using the constructed spectral models to detect the “target” class (e.g., tumor). Here, each time the model is built, all spectra from one patient are left out of the training set of data used to build the model. The data for the patient that is left out is used as the test set.

An important step in building and evaluating the PLSDA model is Partial Least Squares (PLS) factor selection. Retaining excess PLS factors may lead to overfitting of the class/spectra data, which may include systematic noise sources. Retaining too few PLS factors leads to underfitting of the class/spectra data. A confusion matrix may be employed as a Figure of Merit (FOM) for the optimal selection of PLS factors. A misclassification rate for the PLSDA model may be evaluated as a function of the retained PLS factors. However, the misclassification rate, although an important parameter, may not be very descriptive of the final ROC curve, which is the basis for model performance. For example, the misclassification rate is impacted by uneven class sizes, which is a motivation for using other metrics. As such, in some embodiments, an alternative FOM, such as the Area Under the ROC curve (AUROC), Youden's index, F1 score, and/or the minimum distance to an ideal sensor (distance to corner), may be used for the optimal selection of PLS factors.

A model may be built using all patients and an optimal number of factors. A ROC curve may be generated and analyzed. A ROC curve may represent a plot of sensitivity (true positive rate) and 1-specificity (false positive rate) and may be used as a test to select a threshold score that maximizes sensitivity and specificity. The threshold score may correspond to the optimal operating point on the ROC curve that is generated by processing the training data. The threshold score may be selected such that the performance of the classifier is as close to an ideal sensor as possible. An ideal sensor may have a sensitivity equal to 100%, a specificity equal to 100%, an AUROC of 1.0, and may be represented by the upper left corner of the ROC plot. To select the optimal operating point, a threshold may be considered across the observed indices. The true positive, true negative, false positive, and false negative classifications are calculated at each threshold value to yield the sensitivity and specificity results. The optimal operating point is the point on the ROC curve that is the minimum distance from the ideal sensor. The threshold value that corresponds to the maximum sensitivity and specificity may be selected as the threshold value for the model. Additional metrics that could be used may include Youden's index and the F1 score. Alternatively, the threshold can be calculated by using a cluster method, such as Otsu's method. Using Otsu's method, a histogram may be calculated using the scores from the training data, and the histogram may be sub-divided into two parts or classes. The result of applying a threshold to an image may be referred to as a class image.

The two-class model may be applied to the spectrum at each pixel in the hyperspectral image to generate two score images, one corresponding to a characteristic of interest (a target image) and one corresponding to a non-target image. A score between 0 and 1 is assigned to the spectrum associated with each pixel and represents the probability that the tissue at that location is the target. These probabilities may be directly correlated to the intensity of each pixel in a grayscale (e.g., score) image that is generated for each sample. In some embodiments, software, such as Chemlmage Xpert® software, may be used to digitally stain (add coloration) to the score image and create an RGB image (e.g., green=tumor histological subtype 1, blue=non-histological-tumor subtype 1).

In some embodiments, a mask image may be generated. In such embodiments, a region of interest may be selected from the hyperspectral image, and a binary image may be generated from the region of interest. An intensity of one may be used for pixels that correspond to the biological tissue, and an intensity of zero may be used for pixels that do not correspond to the biological tissue (e.g., background pixels). Tumor histological subtype 1 and non-tumor-histological subtype 1 score images may be multiplied by the mask image to eliminate non-relevant pixels. After the non-relevant pixels are eliminated, the image may be digitally stained.

The present disclosure provides several examples for the detection capabilities of the present disclosure using a two-class PLSDA model. In ex vivo examples, tissue samples were obtained immediately after surgical excision and analyzed using the CONDOR™ imaging system available from ChemImage Corporation, Pittsburgh, Pa. Illumination intensity was optimized using a reflectance standard, and hyperspectral images were generated using two LCTFs (one for the VIS region and one for the NIR region).

In an alternative embodiment, hyperspectral images may only be generated at specific wavelengths of interest instead of generating many images over a desired wavelength range. For example, in an embodiment utilizing thin film fixed bandpass filters, a univariate response may be generated in which two wavelengths are measured. A ratiometric image may be generated by applying at least one ratiometric technique (such as wavelength division). In such an embodiment, spectra are not extracted from the hyperspectral image and analyzed.

In some embodiments, a multi-class PLSDA model may be used to discriminate among a plurality of tumor histological subtypes and non-tumors.

### EXAMPLES

### Example 1—MCI Discrimination of Kidney Tumor Histological Subtypes—Two-Class Models

Human ex vivo tumor tissue samples were excised from 18 patients diagnosed with one of four histological subtypes of kidney cancer: clear cell renal cell carcinoma (ccRCC) (n=13), papillary RCC (n=2), chromophobe RCC (n=1), and transitional cell carcinoma (TCC) (n=2). The tissue samples were analyzed using the CONDOR™ imaging system available from Chemlmage Corporation, Pittsburgh, Pa. Each sample was analyzed from multiple perspectives. In other words, spectra of each tumor were extracted from more than one perspective (i.e., field of view (FOV)). Illumination intensity was optimized using a reflectance standard, and hyperspectral images were generated using two LCTFs (one for the VIS region and one for the NIR region). In sum, hyperspectral images of the tissue samples from the various fields of view were generated in the VIS-NIR range from 520 nm to 1050 nm. The generated hypercubes were corrected for instrument response.

A PLSDA was performed leaving one field of view out for cross validation. In this example, a two-class model was built for each tumor histological subtype v. all other tumor histological subtypes. For example, a two-class model was built for ccRCC v. (papillary RCC+chromophobe RCC+TCC). Performance was evaluated on the ROC curve generated from each of the two-class models. 10 spectra were generated for each field of view for each tissue sample.

Based on prior knowledge of the tumor histological subtype for each tissue sample, the sensitivity (true positive), specificity (true negative), accuracy, and AUROC for each model were determined. A number of factors for each model was also determined. The resulting findings are provided in Table 1.

Table 1
Statistical Analysis of Two-Class Models

| | 2-Class | | | | | |
|---|---|---|---|---|---|---|
| Model | PLS-DA Model | Sensi- | Speci- | Accu- | | # |
| # | Description | tivity | ficity | racy | AUROC | Factors |
| 1 | ccRCC (26 FOVs) | 100.0% | 100.0% | 100.0% | 1.000 | 6 |
| | v. All Others | | | | | |
| | (12 FOVs) | | | | | |
| 2 | Chromophobe | 100.0% | 100.0% | 100.0% | 1.000 | 6 |
| | RCC (2 FOVs) | | | | | |
| | v. All | | | | | |
| | Others (36 FOVs) | | | | | |
| 3 | Papillary RCC | 83.3% | 90.6% | 89.5% | 0.896 | 10 |
| | (6 FOVs) v. | | | | | |
| | All Others | | | | | |
| | (32 FOVs) | | | | | |
| 4 | TCC (4 FOVs) v. | 100.0% | 97.1% | 97.4% | 0.993 | 5 |
| | All Others | | | | | |
| | (34 FOVs) | | | | | |

### Example 2—MCI Discrimination of Kidney Tumor Histological Subtypes—Multi-Class Model

Human ex vivo tumor tissue samples were excised from 18 patients diagnosed with one of four histological subtypes of kidney cancer: clear cell renal cell carcinoma (ccRCC) (n=13), papillary RCC (n=2), chromophobe RCC (n=1), and transitional cell carcinoma (TCC) (n=2). The tissue samples were analyzed using the CONDOR™ imaging system available from Chemlmage Corporation, Pittsburgh, Pa. Each sample was analyzed from multiple perspectives, In other words, spectra of each tumor were extracted from more than one perspective (i.e., field of view (FOV)). Illumination intensity was optimized using a reflectance standard, and hyperspectral images were generated using two LCTFs (one for the VIS region and one for the NIR region). In sum, hyperspectral images of the tissue samples from the various fields of view were generated in the VIS-NIR range from 520 nm to 1050 nm. The generated hypercubes were corrected for instrument response.

A PLSDA was performed leaving one field of view out for cross validation. In this example, a four-class model was built using the one-vs-all classification methodology in which each tumor histological subtype comprised its own class. Performance was evaluated on the misclassification rate generated for the four-class model. 10 spectra were generated for each field of view.

Based on prior knowledge of the tumor histological subtype for each tissue sample, the ability of the four-class model to correctly classify the spectra into the proper class was evaluated. The resulting PLS-based confusion matrix for the four-class model is provided in Table 2.

TABLE 2
Confusion Matrix for Multi-Class Model

| | | | | | Misclassification |
|---|---|---|---|---|---|
| | ChromRCC | ccRCC | Papillary | TCC | Rate |
| ChromRCC | 2 | 0 | 0 | 0 | 0% |
| ccRCC | 1 | 21 | 2 | 2 | 19.2% |
| Papillary | 0 | 1 | 4 | 1 | 33.3% |
| TCC | 0 | 0 | 0 | 4 | 0% |
| Average | | | | | 18.4% |

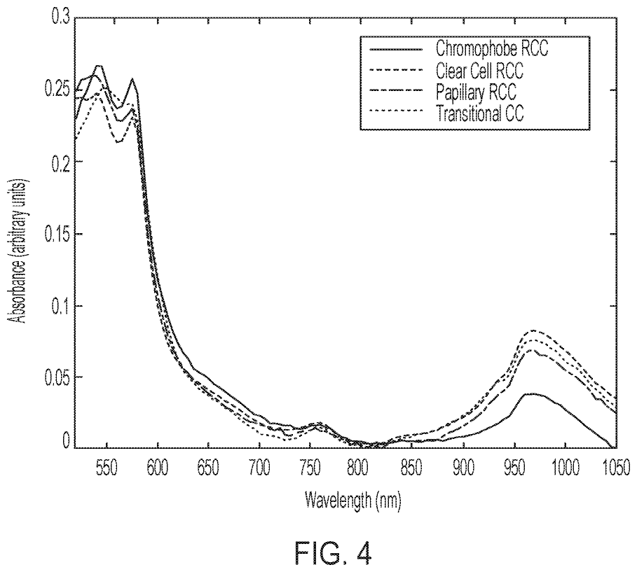

FIG. 4 depicts average VIS-NIR spectra for each class (i.e., tumor histological subtype). As shown in FIG. 4, identifiable differences in the absorbance rate exist at a plurality of wavelengths among the tissues for the four kidney cancer tumor histological subtypes.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of analyzing biological tissue, the method comprising:
illuminating the biological tissue to generate a plurality of interacted photons;
collecting the plurality of interacted photons;
detecting the plurality of interacted photons to generate at least one hyperspectral image;
analyzing the at least one hyperspectral image by extracting a spectrum from a location in the at least one hyperspectral image, wherein the location corresponds to an area of interest of the biological tissue; and
analyzing the extracted spectrum to differentiate a tumor histological subtype present within the biological tissue.

2. The method of claim 1, wherein the biological tissue comprises tissue from one or more of a kidney, a ureter, a prostate, a penis, a testicle, a bladder, a heart, a brain, a liver, a lung, a colon, an intestine, a pancreas, a thyroid, an adrenal gland, a spleen, a stomach, a uterus, and an ovary.

3. The method of claim 1, wherein the tumor histological subtype comprises a histological subtype of one or more of kidney cancer, bladder cancer, bone cancer, brain cancer, breast cancer, colon cancer, intestinal cancer, liver cancer, lung cancer, ovarian cancer, pancreatic cancer, prostate cancer, rectal cancer, skin cancer, stomach cancer, testicular cancer, thyroid cancer, urethral cancer, and uterine cancer.

4. The method of claim 1, further comprising generating a bright-field image representative of the biological tissue.

5. The method of claim 4, further comprising analyzing the bright-field image to identify one or more of a morphological feature of the biological tissue and an anatomical feature of the biological tissue.

6. The method of claim 1, wherein analyzing the extracted spectrum further comprises comparing the extracted spectrum to a reference spectrum associated with a known characteristic.

7. The method of claim 6, wherein the comparing comprises applying an algorithmic technique.

8. The method of claim 7, wherein the algorithmic technique comprises one or more of a multivariate curve resolution analysis, a principle component analysis (PCA), a partial least squares discriminant analysis (PLSDA), a non-negative matrix factorization, a k means clustering analysis, a band target entropy method analysis, an adaptive subspace detector analysis, a cosine correlation analysis, a Euclidian distance analysis, a partial least squares regression analysis, a spectral mixture resolution analysis, a spectral angle mapper metric analysis, a spectral information divergence metric analysis, a Mahalanobis distance metric analysis, and a spectral unmixing analysis.

9. The method of claim 7, wherein the algorithmic technique comprises one or more of a support vector machine and a relevance vector machine.

10. The method of claim 7, wherein the algorithmic technique is applied to spectra corresponding to each pixel of the at least one hyperspectral image to generate at least one score image.

11. The method of claim 10, wherein the at least one score image comprises one or more of a target image and a non-target image.

12. The method of claim 11, further comprising applying a threshold to the target image to generate a class image of the biological tissue.

13. The method of claim 10, further comprising generating an RGB image of the biological tissue, wherein at least one channel of the RGB image corresponds to the target image.

14. The method of claim 10, further comprising generating an RGB image of the biological tissue, wherein at least one channel of the RGB image corresponds to a non-target image.

15. The method of claim 1, wherein the hyperspectral image comprises a VIS-NIR hyperspectral image.

16. The method of claim 1, wherein the hyperspectral image comprises a SWIR hyperspectral image.

17. The method of claim 1, further comprising passing the plurality of interacted photons through a filter to filter the interacted photons across a plurality of wavelength bands.

18. A system for analyzing biological tissue, the system comprising one or more processors coupled to a non-transitory processor-readable medium, the non-transitory processor-readable medium including instructions that, when executed by the one or more processors, cause the system to:
illuminate the biological tissue to generate a plurality of interacted photons;
collect the plurality of interacted photons;
detect the plurality of interacted photons to generate at least one hyperspectral image;
analyze the at least one hyperspectral image by extracting a spectrum from a location in the at least one hyperspectral image, wherein the location corresponds to an area of interest of the biological tissue; and
analyze the extracted spectrum to differentiate a tumor histological subtype present within the biological tissue.

19. The system of claim 18, wherein the biological tissue comprises tissue from one or more of a kidney, a ureter, a prostate, a penis, a testicle, a bladder, a heart, a brain, a liver, a lung, a colon, an intestine, a pancreas, a thyroid, an adrenal gland, a spleen, a stomach, a uterus, and an ovary.

20. The system of claim 18, wherein the tumor histological subtype comprises a histological subtype of one or more of kidney cancer, bladder cancer, bone cancer, brain cancer, breast cancer, colon cancer, intestinal cancer, liver cancer, lung cancer, ovarian cancer, pancreatic cancer, prostate cancer, rectal cancer, skin cancer, stomach cancer, testicular cancer, thyroid cancer, urethral cancer, and uterine cancer.

21. The system of claim 18, wherein the instructions, when executed by the one or more processors, further cause the system to generate a bright-field image representative of the biological tissue.

22. The system of claim 21, wherein the instructions, when executed by the one or more processors, further cause the system to analyze the bright-field image to identify one or more of a morphological feature of the biological tissue and an anatomical feature of the biological tissue.

23. The system of claim 18, wherein the instructions, when executed by the one or more processors, further cause the system to compare the extracted spectrum to a reference spectrum associated with a known characteristic.

24. The system of claim 23, wherein the comparing comprises applying an algorithmic technique.

25. The system of claim 24, wherein the algorithmic technique comprises one or more of a multivariate curve resolution analysis, a principle component analysis (PCA), a partial least squares discriminant analysis (PLSDA), a non-negative matrix factorization, a k means clustering analysis, a band target entropy method analysis, an adaptive subspace detector analysis, a cosine correlation analysis, a Euclidian distance analysis, a partial least squares regression analysis, a spectral mixture resolution analysis, a spectral angle mapper metric analysis, a spectral information divergence metric analysis, a Mahalanobis distance metric analysis, and a spectral unmixing analysis.

26. The system of claim 24, wherein the algorithmic technique comprises one or more of a support vector machine and a relevance vector machine.

27. The system of claim 24, wherein the instructions, when executed by the one or more processors, further cause the system to apply the algorithmic technique to spectra corresponding to each pixel of the at least one hyperspectral image to generate at least one score image.

28. The system of claim 27, wherein the at least one score image comprises one or more of a target image and a non-target image.

29. The system of claim 28, wherein the instructions, when executed by the one or more processors, further cause the system to apply a threshold to the target image to generate a class image of the biological tissue.

30. The system of claim 28, wherein the instructions, when executed by the one or more processors, further cause the system to generate an RGB image of the biological tissue, wherein at least one channel of the RGB image corresponds to the target image.

31. The system of claim 28, wherein the instructions, when executed by the one or more processors, further cause the system to generate an RGB image of the biological tissue, wherein at least one channel of the RGB image corresponds to a non-target image.

32. The system of claim 18, wherein the hyperspectral image comprises a VIS-NIR hyperspectral image.

33. The system of claim 18, wherein the hyperspectral image comprises a SWIR hyperspectral image.

34. The system of claim 18, wherein the instructions, when executed by the one or more processors, further cause the system to pass the plurality of interacted photons through a filter to filter the interacted photons across a plurality of wavelength bands.
